(19) United States
(12) Patent Application Publication
(10) Pub. No.: US 2010/0143508 A1
(43) Pub. Date: Jun. 10, 2010

(54) Limonene and yucca medical compound for treating human diseases and conditions

(76) Inventors: Bill T. Scarbrough, Porterville, CA (US); Huu S. Tieu, Porterville, CA (US)

Correspondence Address: RICHARD A. RYAN;ATTORNEY AT LAW, 8497 N. MILLBROOK AVENUE, SUITE 101, FRESNO, CA, 93720, US

(21) Appl. No.: 12/381,923

(22) Filed: Mar. 18, 2009

Related U.S. Application Data
(60) Provisional application No. 61/201,022, filed on Dec. 5, 2008.

(51) Int. Cl. A61K 36/00; A61K 31/015; A61P 35/00

(52) U.S. Cl. 424/725; 514/763

(57) ABSTRACT

A medical compound for treating a wide variety of human diseases and conditions comprises a medically effective amount of a yucca extract, an effective delivery-enhancing amount of d-Limonene and an organic solvent as the active components. The inactive components include one or more inert solvents and an inert emulsifier. In the preferred embodiment, the yucca extract is Sarsasapogenin at 45% by volume, the d-Limonene is in an amount of 19.1% by volume and the organic solvent is 2-Butoxyethanol at 13.5% by volume. The inert components, making up the rest of the compound, are the inert solvent isoparaffinic fluid, the inert emulsifier sodium dodecylbenzene sulfonate and the inert solvent isopropylamine. The medical compound has a unique delivery system that readily enters the systemic circulation and rapidly reaches the target tissue and/or organs to quickly begin the process of restoring and healing the patient by boosting his or her immune system.

### CROSS-REFERENCE TO RELATED APPLICATIONS

This patent application claims priority to U.S. Provisional Patent Application No. 61/201,022 filed Dec. 5, 2008.

### STATEMENT REGARDING FEDERALLY SPONSORED RESEARCH

Not Applicable.

### BACKGROUND OF THE INVENTION

A. Field of the Invention

The field of the present invention relates to medical compounds for treating human diseases and other conditions. More particularly, the present invention relates to such compounds that comprise limonene and yucca to treat various human diseases and conditions, including cancer, cardiovascular diseases, herpes, inflammatory conditions, infections, HIV, Alzheimer disease, urinary problems, hair growth and asthma. Even more particularly, the present invention relates to such compounds that include a delivery system which quickly and effectively delivers the medically effective components to the parts of the body needing treatment.

B. Background

Many persons are investigating the potential of treating a variety of human diseases and other conditions with more natural and/or herbal compounds to reduce the side effects and inefficiencies of treating patients with conventional drugs. For instance, typical cancer treatments comprise the use of radiation therapy and chemotherapy, which are both known to be invasive and have a variety of debilitating effects on the patient. Diseases such as HIV and AIDS are primarily treated with an anti-viral cocktail that is generally considered somewhat experimental and palliative. Diabetes, both type I and II, are typically treated with invasive insulin injection and Sulfonylurea pills. Other diseases, including Alzheimer's, Parkinson's, Cerebral Palsy, Dermatomyositis and Systemic Lupus (SLE) have no known cure and/or no known effective treatment. Due to the problems associated with current treatment methods and compounds or the lack of such treatment methods and compounds there is a desire to identify natural and organic compounds to treat various diseases and conditions. Three such compounds are d-limonene, yucca plant extract and 2-Butoxyethanol (an organic solvent).

The chemical compound d-limonene has been around for many years and has been proven to be safe for use with plants, animals and humans. This chemical is a major constituent of citrus oils, including those obtained from orange, lemon, lime and grapefruit, extracted from citrus rinds or peels. During the processing of citrus fruits into juice, the citrus oil is pressed out of the rind. This citrus oil is separated from the juice and then distilled to recover certain flavor and fragrance compounds. The bulk of the oil, however, is left behind and collected, which is food grade d-limonene. After the juicing process, the rinds are conveyed to a steam extractor to extract more oil from the rind. When the steam is condensed, a layer of citrus oil floats on the surface of the condensed water, providing technical grade d-limonene. It is well known that, in addition to being a safe chemical, d-limonene can be utilized with other chemicals to provide compositions which are effective as a fungicide, bactericide, pesticide and other uses. For instance, U.S. Pat. No. 3,023,144 to Greathouse, et al. describes d-limonene based compounds configured as topical agents for human and animal use in the form of ointments, lotions, creams, shampoos and similar products. U.S. Pat. No. 3,960,539 to Newhall discloses d-limonene use in a compound for the regulation of plant growth and the control of nematodes and fungi on plants. U.S. Pat. No. 4,379,168 to Dotolo describes the use of d-limonene in a pesticide composition that also contains a surfactant or emulsifier in water to form a composition that is non-toxic and non-irritating to animals. U.S. Pat. No. 6,849,276 to Dufau, et al. describes a liquid composition having a monocyclic terpenic hydrocarbon, such as limonene, that has fungicide, bactericidal and bacteriostatic activity with the copper maintained in suspension in the aqueous emulsion.

Yucca plants include a number of different trees and shrubs found in arid portions of North and Central America. Common species include Yucca aloifolia (Spanish bayonet), Y. brevifolia (Joshua tree), Y. filamentosa (Adam's needle), Y. glauca (soap-weed), and many others. All parts of the plant, as well as many different species, are used. Yucca is used as a dietary supplement in the United States and is commonly marketed as an anti-inflammatory herb, primarily for the treatment of arthritis symptoms. There are also claims that yucca may help reduce blood pressure and cholesterol levels. Traditionally, yucca has been used by different cultures for a wide variety of medical conditions, including gout, gall bladder problems, diabetes, genitourinary disorders, indigestion, and constipation, and also has been used as a diuretic and topically for inflammation or general skin cleansing. Native Americans have created soap, shampoo, rope, and textiles from yucca plants. Plant constituents are also used commercially as foaming agents and flavorings. Yucca plants contain steroidal saponins such as sarsasapogenin and tigogenin. Saponins are widely used for their detergent and foaming properties, and have also been studied in animals for their potential anticholesterol, anti-inflammatory, and anticarcinogenic activities. Yucca leaf protein can interfere in vitro with the protein synthesis of cells infected by herpes simplex virus and cytomegalovirus. Flowers of certain yucca species contain polysaccharides with tumor-inhibiting effects in mice. In general, saponins may cause dose-dependent gastrointestinal distress, especially in raw plant form. In controlled clinical trials using yucca tablets, mild and transient complaints were reported in about 9% of patients, and unfavorable gastrointestinal effects were reported in 4%. Native Americans and others have used the yucca plant as food for centuries without any known adverse effects. Precise doses for yucca have not been established. In the clinical studies that used a yucca saponin extract, tablets were taken three times daily, usually with or after meals. The amount of yucca or saponin in each tablet, and other ingredients in the extract, were not described. Traditionally, herbalists have made decoctions by boiling the roots or young shoots in water.

One yucca plant is Yucca schidigera, an herbaceous plant of the lily family native to the deserts of the south-western United States and northern Mexico. This plant was used in traditional medicine by Native Americans to treat a variety of ailments including arthritis. Yucca products are currently used in a number of applications. Yucca powder and yucca extract are used as animal feed additives. Beneficial effects in livestock and poultry production include increased growth rate and improved feed conversion efficiency, reduction in atmospheric ammonia in confinement animal and poultry facilities, anti-protozoal and nematocidal activity, modification of ruminal microbe populations, inhibition of Gram-positive bacteria, reductions in stillbirths in swine, reduction in egg and tissue cholesterol contents, and anti-arthritic activity in horses and dogs. Other applications include the use of yucca extract as a foaming agent in beverages, and use in crop production as nematode and fungi-control agents, as a soil wetting agent, and crop growth stimulant. Yucca products have GRAS status, so are FDA-approved for use in humans.

2-Butoxyethanol is an organic solvent with the formula C₆H₁₄O₂. The chemical is a colorless liquid with a sweet, ether-like odor. It is a butyl ether of ethylene glycol. The main use of 2-Butoxyethanol is as a solvent in paints and surface coatings, followed by cleaning products and inks. Other products which contain 2-Butoxyethanol include acrylic resin formulations, asphalt release agents, firefighting foam, leather protectors, oil spill dispersants, bowling pin and lane degreaser, and photographic strip solutions. 2-Butoxyethanol is a primary ingredient of various whiteboard cleaners, liquid soaps, cosmetics, dry cleaning solutions, lacquers, varnishes, herbicides, and latex paints. It also seems to be excellent at killing most insects and arachnids. This chemical is also the main ingredient of many home, commercial, and industrial cleaning solutions. The chemical 2-Butoxyethanol usually decomposes in the environment within a few days and has not been identified as a major environmental contaminant. It is not known to build up in any plant or animal species.

The immune system of the human body is known to be able to resist many types of diseases and “treat” diseases and other medical conditions without the addition of many of the medicines that are commonly utilized today and prescribed by the medical community. Often, however, for a variety of reasons a person's immune system is weakened or otherwise unable to effectively respond to an attach against the body by a disease or other medical condition. What is needed therefore is a new compound for boosting a person's immune system so his or her body can more effectively resist and/or respond to diseases and/or medical conditions. The new compound should be effectively treat a variety of diseases and medical conditions by boosting a person's own immune system. Preferably, the new compound should be safe to use and have few, if any, side effects to reduce or eliminate the need for secondary or supplemental medication to address such side effects. The new compound should include an effective delivery system that directs the medically effective components of the compound to the parts of the human body which need treatment.

### SUMMARY OF THE INVENTION

The limonene and yucca medical compound for treating human diseases and conditions of the present invention provides the treatment benefits and solves many of the problems associated with conventional treatments. In a preferred embodiment of the compound and method of the present invention, d-Limonene and a yucca extract are combined with 2-Butoxyethanol, as well as various inert components, to form a medical compound that treats a variety of different human diseases and conditions, as set forth below. The compound of the present invention effectively and safely boosts a person's immune system so his or her body can more effectively resist and/or respond to various diseases and/or medical conditions. The compound of the present invention has no known ill or side effects, thereby eliminating the need for secondary or supplemental medication to address such side effects. In its preferred use, the compound is administered orally, preferably sub-lingual, which eliminates the need for use of invasive administration techniques. In the preferred configuration, the compound has a unique and effective delivery system that allows the compound to readily enter the human body's systemic circulation to rapidly reach the target area where the treatment is needed. Because the compound of the present invention is readily absorbed, it will accomplish the objective of restoring and healing the patient in a very short period of time.

In a general aspect of the present invention, the medical compound comprises a mixture of a yucca extract, d-Limonene and an organic solvent as the active components and one or more inert solvents, surfactants and/or emulsifiers as the inert components. In the preferred embodiment of the present invention, the yucca extract is Sarsasapogenin and the organic solvent is 2-Butoxyethanol. In a preferred embodiment, the inert components comprise isoparaffinic hydrocarbon fluid as the inert solvent and Sodium dodecylbenzene sulfonate as the inert emulsifier emulsifier. The preferred configuration of the present medical compound also contains Isopropylamine as an additional inert solvent. In a preferred embodiment, the Sarsasapogenin is present in an approximate amount ranging between ______% to ______% by volume, the d-Limonene is present in an approximate amount of ______% to ______% by volume and the 2-Butoxyethanol is present in an approximate range of ______% to ______% by volume, with the various inert components making up the difference. In the preferred embodiment of the present invention, the medical compound comprises approximately 45% (by volume) of Sarsasapogenin, approximately 19% d-Limonene (by volume) and approximately 13.5% 2-Butoxyethanol (by volume), with the inert compounds comprising the other approximately 22.5% (by volume).

Accordingly, the primary objective of the present invention is to provide a medical compound for treating various human diseases and medical conditions that provides the advantages discussed above and that overcomes the disadvantages and limitations associated with presently available compounds and methods for treating such diseases and medical conditions.

It is an important objective of the present invention to provide a medical compound for treating human diseases and conditions that boosts a person's immune system so his or her body can more effectively resist and/or respond to diseases and/or medical conditions.

It is also an important objective of the present invention to provide a medical compound comprising d-Limonene, a yucca extract and 2-Butoxyethanol as the active components to safely and effectively treat a variety of different human diseases and conditions by boosting the immune system without side effects that would otherwise require the need for secondary or supplemental medication to address.

Another important objective of the present invention is to provide a medical compound comprising d-Limonene, a yucca extract and 2-Butoxyethanol as the active components that can be administered orally, preferably sub-lingual, so as to eliminate the need to utilize invasive administration techniques.

Yet another important objective of the present invention is to provide a medical compound having d-Limonene, Sarsasapogenin and 2-Butoxyethanol as the active components to provide the compound with an unique and effective delivery system that allows the medical compound to readily enter the body's systemic circulation system and rapidly reach the target area where it is needed so that it may restore and heal the patient in a relatively short period of time.

The above and other aspects and advantages of the present invention are explained in greater detail by reference to the attached figures and the description of the preferred embodiment which follows. As set forth herein, the present invention resides in the novel features of form, construction, mode of operation and combination of the above presently described and understood by the claims.

### DETAILED DESCRIPTION OF THE PREFERRED EMBODIMENTS

With reference to the various preferred embodiments of the present invention set forth below, these embodiments are intended to be representative of one or more ways of configuring the medical compound of the present invention. Although specific chemicals, materials, configurations and uses are set forth herein, it should be understood that a number of variations to the components described herein can be made without changing the scope and function of the present invention. For instance, although the description provided herein sets forth certain components and percentages of those components that make-up the preferred embodiments of the medical compound of the present invention, those who are skilled in the art will readily understand that this is merely for purposes of simplifying the present disclosure and that the present invention is not so limited. Specifically, as will be readily appreciated by those skilled in the art, various equivalent chemicals can be utilized to make-up the medical compound of the present invention.

In a preferred embodiment, the medical compound of the present invention comprises a medically effective amount of yucca extract, an effective delivery-enhancing amount of d-Limonene (CAS # 5989-27-5), and an organic solvent, as well as one or more inert solvents and inert emulsifiers. In the preferred embodiment, the active components of the medical compound of the present invention comprises the yucca extract Sarsasapogenin, the d-Limonene and the organic solvent 2-Butoxyethanol. The inactive components of the preferred embodiment comprise the inert solvent Isoparaffinic fluid, the inert emulsifier Sodium dodecylbenzene Sulfonate and a small amount of the inert solvent Isopropylamine. The yucca extract Sarsasapogenin is the absolute stereochemistry of the C-25 carbon, which is S, with the formula C₂₇H₄₄O₃ (CAS # 126-19-2) and is commercially available from Cell-U-Con out of Strathmore, Calif. as Therm X-70 (CAS # 90147-57-2). A number of processors, such as Florida Chemical Company out of Winter Haven, Fla., supply d-Limonene (CAS # 8028-48-6) suitable for use with the medical compound of the present invention. In a preferred composition of the medical compound of the present invention, the 2-Butoxyethanol component is Butyl Cellusolve (CAS # 111-76-2), which is available from Univar Corporation out of Seattle, Wash. With regard to the inert components, the Isoparaffinic fluid inert solvent is Isopar-M (CAS # 64742-47-8) available from Exxon-Mobile Corporation out of Fairfax, Va., the Sodium dodecylbenzene Sulfanate inert emulsifier is Calsoft L-60 (CAS # 68081-81-2 and 1300-72-7) available from Pilot Chemical Company out of Cincinnati, Ohio and the Isopropylamine (CAS # 75-31-0, 68584-24-7 and 68584-87-3) is Calimulse PRS available from the Pilot Chemical Company. As will be readily apparent to those skilled in the art, various equivalent chemicals and chemical compositions can be substituted for those identified above and effectively accomplish the objectives of the medical compound of the present invention set forth herein.

A preferred embodiment of the medical compound of the present invention is set forth in the formulation below:

| | APPROXIMATE | PREFERRED | |
|---|---|---|---|
| CHEMICAL | % BY VOLUME | RANGE | USE |
| Sarsasapogenin | 45.00 | 35.00 to 55.00 | Active |
| d-Limonene | 19.10 | 15.00 to 25.00 | Active |
| 2-Butoxyethanol | 13.50 | 8.00 to 16.00 | Active |
| Isoparaffinic fluids | 5.90 | 4.00 to 7.00 | Inert solvent |
| Sodium | 16.20 | 14.00 to 18.00 | Inert Emulsifier |
| dodecylbenzene | | | |
| Sulfonate | | | |
| Isoproplyamine | <0.10 | — | Inert solvent |

The unique delivery system of the medical compound of the present invention is substantially responsible for the benefits of the medical compound, which results in the complex component of the invention reaching the systemic circulation in a chemically unchanged form. As such, the medical compound of the present invention has a bioavailability of nearly one hundred percent. The three active components of the medical compound show bio-equivalence and therapeutic equivalence. In the preferred embodiment, the medical compound is prepared in an oral liquid and/or a gel capsule form that is administered to the patient by mouth, preferably sublingually, utilizing liquid drops. The medical compound reaches the systemic circulation very rapidly, nearly instantaneously, bypassing the so called “First Pass Hepatic Metabolosim” effect. Although the medical compound has some solubility in an aqueous solution, the solubility of the medical compound is primarily hydrophobic, thereby allowing it to be well absorbed. Additionally, the medical compound has the capability to also cross the “Blood-Brain Barrier,” thereby benefitting neurological conditions.

The medical compound of the present invention accomplishes its objective of beneficially treating various human diseases and conditions by repairing, restoring and then healing the damaged tissue and organs, which is achieved through the extraordinary delivery system of the medical compound. The physical and chemical properties of the medical compound begin to act on the damaged tissue and organs as soon as it reaches the target areas. After the medical compound as secured the disease or condition, it is anticipated that maintenance amounts of the medical compound will be necessary for continued health benefits.

The medical compound of the present invention incorporates the nuclear transcription factor KappaB (NF-KB) inhibitor that regulates and suppresses the pathways of genes for mutagenic growth, viral replication, inflammation, bactericidal, neoplastic and autoimmune diseases. The medical compound of the present invention is highly mobile in the human body and is active in the central nervous system disorders. The d-Limonene and yucca extract Sarsasapogenin (sarsasapogenin glycoside) are approved for use in foods and beverages and are generally recognized as safe (GRAS) rated by the United States Food and Drug Administration under Title 21, CFR 172.510, Flavor and Extract Manufactures Association (FEMA) number 3121.

As an immune system booster, the medical compound of the present invention has several immediate and intermediate benefits as well as several observed curative properties. The immediate benefits include an increase in energy, alertness and appetite, improved restful sleep and bowel regulation. The intermediate benefits realized by the medical compound of the present invention include improved general well-being, reduction in anxiety, psychosis and memory loss and improved socialization. The observed curative properties of the medical compound of the present invention include anti-neoplastic, anti-inflammatory, anti-oxidant, anti-bacterial, anti-fungal, anti-parasitic, anti-viral and analgesic. The medical compound of the present invention has the following observed benefitted (diagnosed) conditions:

1. Infectious Diseases: HIV/AIDS, hepatitis C, tuberculosis, herpes, measles and shingles.

2. Cancer: Malignant neoplasm, malignant tumor, carcinoma, sarcoma, lymphoma, leukemia and germ cell tumor. In addition, the medical compound of the present invention has also been found to shorten the recovery phase of chemotherapy and radiation therapy for cancer patients.

3. Neurological: Cerebral Palsy, Alzheimer's disease, Parkinson's disease, dementia, epilepsy, psychosis, attention-deficit disorder, depression and seizures.

4. Endocrine: Thyroiditis, hyperthyroidism, hypothyroidism and diabetes type I and II.

5. Cardiovascular: Hypertension, hyperlipidemia, stroke, thrombosis and hypotension.

6. Respiratory: Asthma, allergy, sinusitis, bronchitis, emphysema and pneumonia.

7. Immune System: Arthritis, Osteoarthritis, dermatomyositis, systemic lupus (SLE) and renal diseases.

The medical compound of the present invention is manufactured by mixing them in order, beginning with d-Limonene, Isopropylamine (PRS), Sodium dodecylbenzene Sulfanate (L-60), 2-Butoxyethanol (EB), Isoparaffinic fluid (Isopar) and the yucca extract (Sarsasapogenin/Therm X-70). The first three components must be added in the order set forth above at sixty-five degrees Fahrenheit (65° F.) or above to prevent the separation of the MF-450. The Therm X-70 is added only after the Limonene concentrate formulation is complete and test for correct characteristics. All ingredients in this product are tech grade and no reaction occurs when formulating and no resulting impurities have been found in a bath test or in a test conducted at three months intervals. The medical compound of the present invention should be manufactured utilizing established Good Laboratories Practice. The resulting medical compound of the present invention is far superior to products that are currently available. The physical properties of the medical product include having a citrus smell, a taste that is tangy and mildly bitter, suitable for room temperature storage and has a shelf-life of approximately one year after the manufacturing date. The inherent beneficial properties of the medical compound of the present invention are achieved with a natural (e.g., non-synthetic) product that is ubiquitous in nature. The benefits of the medical compound of the present invention are applicable to pediatric, adult and geriatric patients. In one embodiment, the medical compound is taken orally, preferably by applying 0.25 to 0.75 ml drops of the liquid solution under the tongue one to five times a day.

While there are shown and described herein specific forms of the invention, it will be readily apparent to those skilled in the art that the invention is not so limited, but is susceptible to various modifications and rearrangements in materials without departing from the spirit and scope of the present invention. In particular, it should be noted that the present invention is subject to modification with regard to any the combination of materials and the prescribed use of the medical compound. For instance, there are numerous components described herein that can be replaced with equivalent functioning components to accomplish the objectives of the present invention.

What is claimed is:

1. A medical compound, comprising a medically effective amount of yucca extract, an effective delivery-enhancing amount of d-Limonene and an effective amount of an organic solvent.

2. The medical compound of claim 1 further comprising one or more inert solvents and an inert emulsifier.

3. The medical compound of claim 2, wherein said one or more inert solvents includes an Isoparaffinic hydrocarbon fluid.

4. The medical compound of claim 3, wherein said inert emulsifier is Sodium dodecylbenzene Sulfonate.

5. The medical compound of claim 4, wherein said one or more inert solvents further comprises Isopropylamine.

6. The medical compound of claim 1, wherein said medical compound is a liquid.

7. The medical compound of claim 1, wherein said yucca extract is Sarsasapogenin.

8. The medical compound of claim 7, wherein said organic solvent is 2-Butoxyethanol.

9. The medical compound of claim 8, wherein said Sarsasapogenin is present in a range of approximately 35% to 55% by volume, said d-Limonene is present in a range of approximately 8% to 16% by volume and said 2-Butoxyethanol is present in a range of approximately 15% to 25% by volume.

10. The medical compound of claim 9, wherein said d-Limonene is present at approximately 19% by volume, said Sarsasapogenin is present at approximately 45% by volume and said 2-Butoxyethanol is present at approximately 13.5% by volume.

11. The medical compound of claim 1, wherein said organic solvent is 2-Butoxyethanol.

12. The medical compound of claim 1, wherein said medical compound is a tablet.

13. A medical compound, comprising a medically effective amount of Sarsasapogenin, an effective delivery-enhancing amount of d-Limonene, an effective amount of an organic solvent, one or more inert solvents and an inert emulsifier.

14. The medical compound of claim 13, wherein said one or more inert solvents includes an Isoparaffinic hydrocarbon fluid and said inert emulsifier is Sodium dodecylbenzene Sulfonate.

15. The medical compound of claim 14, wherein said one or more inert solvents further comprises Isopropylamine.

16. The medical compound of claim 13, wherein said organic solvent is 2-Butoxyethanol.

17. The medical compound of claim 16, wherein wherein said Sarsasapogenin is present in a range of approximately 35% to 55% by volume, said d-Limonene is present in a range of approximately 8% to 16% by volume and said 2-Butoxyethanol is present in a range of approximately 15% to 25% by volume.

18. A medical compound, comprising a medically effective amount of Sarsasapogenin, an effective delivery-enhancing amount of d-Limonene, an effective amount of 2-Butoxyethanol, one or more inert solvents and an inert emulsifier, said inert solvents comprising an Isoparaffinic hydrocarbon fluid and said inert emulsifier comprising Sodium dodecylbenzene Sulfonate.

19. The medical compound of claim 18, wherein said Sarsasapogenin is present in a range of approximately 35% to 55% by volume, said d-Limonene is present in a range of approximately 8% to 16% by volume, said 2-Butoxyethanol is present in a range of approximately 15% to 25% by volume, said inert solvents are present in the ranger of approximately 4% to 7% and said inert emulsifier is present in the range of approximately 14% to 18%.

20. The medical compound of claim 19, wherein said one or more inert solvents further comprises Isopropylamine.
